(19) United States
(12) Patent Application Publication
(10) Pub. No.: US 2009/0126108 A1
(43) Pub. Date: May 21, 2009

(54) Oscillating Bed Supporting Structure

(76) Inventor: Clas Rosenbaum, Täby (SE)

Correspondence Address: BIRCH STEWART KOLASCH & BIRCH, PO BOX 747, FALLS CHURCH, VA, 22040-0747, US

(21) Appl. No.: 11/920,930

(22) PCT Filed: Jun. 13, 2006
(86) PCT No.: PCT/SE2006/000702

§ 371 Date: Nov. 21, 2007

(30) Foreign Application Priority Data
Jun. 16, 2005 (SE) 0501369-3

(51) Int. Cl. A61H 23/02; A47C 31/00; A61M 21/00; A61H 7/00; A47C 21/00; A47C 19/02

(52) U.S. Cl. 5/412; 601/90

(57) ABSTRACT

An oscillating bed supporting structure (10) particularly adapted to provide conditions for a snore-free sleep, said structure comprising a base member (11) and a bed supporting member (12) spaced above said base member (11). At least a first oscillation imparting drive means (14) is provided between the base member (11) and the bed supporting member (12) so as to impart to the bed supporting member (12) oscillating movements (A) at least in a substantially horizontal plane.

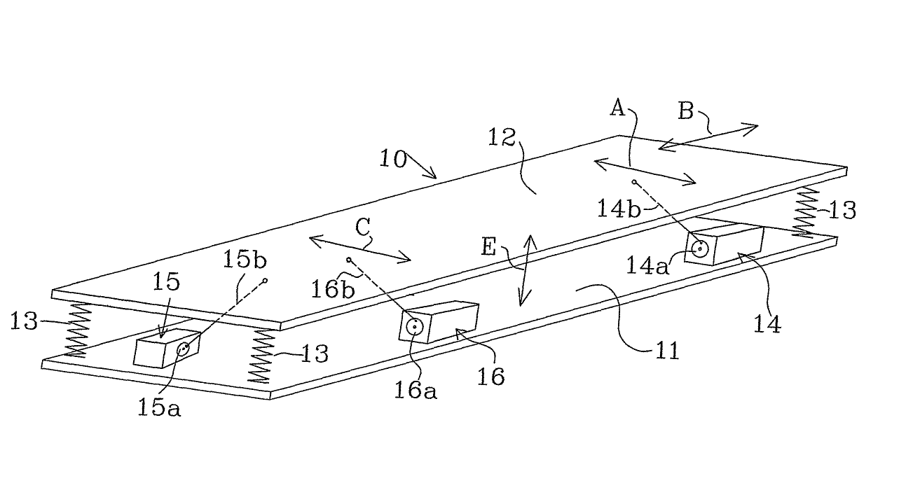

The present invention concerns an oscillating bed supporting structure. It is particularly adapted to provide conditions for a snore-free sleep, but it is believed that it would serve as well as a remedy for other insomnia problems of adults as well as it would serve to rock babies to sleep. It is also likely that it would reduce the risk of bedsores.

The invention is based on observations that people normally disposed to heavy snoring do not snore, or, stop snoring, when sleeping in a moving train. These observations include the observation that—in some instances—snoring is resumed when a train stops, and ceases when the train starts moving again.

DE 38 36292 describes a “Bed for snorers”. According to the Abstract of the patent, a superficial mattress support, which can be pivoted about a longitudinal axis from the horizontal into an inclined position by a controllable drive, rolls a snoring sleeper automatically into the lateral position without waking him, so that he stops snoring. Since the bed can be pivoted back into the horizontal after a short time, undisturbed and relaxed sleep is said to be guaranteed for the snorer.

Evidently, this known bed allows only one repeated monotonous rocking motion possibly interrupted by rest intervals.

As opposed to the patent referred to, the present invention has as its object to provide a bed supporting structure that imitates the mainly randomly occurring oscillating motions of, e.g., a moving train (or other vehicle), at least in a substantially horizontal plane, but preferably to some extent also in a vertical plane, i.e., a three-dimensional movement.

An oscillating bed supporting structure fulfilling this object comprises a base member and a bed supporting member spaced above said base member, at least a first oscillation imparting drive means being provided between said base member and said bed supporting member so as to impart to said bed supporting member an oscillating movement at least in a substantially horizontal plane.

Preferably, there is provided a first and a second separate oscillation imparting drive means imparting to said bed supporting member oscillating movements in mutually substantially perpendicular directions.

Even more preferred, there is provided a third separate oscillation imparting drive means imparting to said bed supporting member oscillating movements in the same direction as one of said first and second oscillation imparting drive means. In such instance, said one and said third oscillation imparting means impart oscillating movements to said bed supporting member at spaced locations along a longitudinal direction of said bed supporting member.

An embodiment of the present invention will now be described, reference being made to the accompanying drawing showing a schematic perspective view of a bed supporting structure.

A bed supporting structure 10 according to the present invention is shown to comprise a substantially horizontal and rectangular base member 11 having the dimensions of any normal bed, such as typically one by two metres. The base member 11 supports a likewise rectangular bed supporting member 12. The bed supporting member is shown here to be supported by four springs 13, only three of which being seen in the drawing. The bed supporting member is adapted to support—in turn—a non-shown mattress or the like, on which a person may rest.

According to a preferred embodiment of the present invention, there are provided three oscillation imparting drive means between the base member 11 and the bed supporting member 12. A first such drive means 14 is located in an area of the bed supporting structure 10 substantially corresponding to the shoulder area of a person resting thereon. It imparts to the bed supporting member 12 an oscillating movement in the directions of arrow A. A second drive means 15 is located in the foot area of a resting person. It imparts to the bed supporting member 12 an oscillating movement in the directions of arrow B. A third drive means 16 is located substantially at the lower part area of the body of a resting person. It imparts to the bed supporting member 12 an oscillating movement in the directions of arrow C.

Each drive means includes a crank mechanism 14a, 15a and 16a, respectively, controllably driven by a non-shown drive motor. The crank mechanisms and their respective drive motors are secured to the base member 11. Each crank mechanism carries a first end of a crank arm 14b, 15b and 16b, respectively, the second, free end of which is attached at a respective different location to the underside of the bed supporting member 12 so as to transfer longitudinal movement of the respective crank arm thereto.

The crank length of each crank mechanism may be individually varied in a non-shown manner evident to the skilled person, so as to provide an amplitude of oscillation typically varying between 1 cm and 5 cm. Also, by varying the revolutionary speed of the respective drive motor in a manner evident to the skilled person, the frequency of oscillation in each direction may be individually varied. By suitable combination of different stroke lengths and different frequencies, the latter preferably controlled by a non-shown computer, simulation of random oscillations may easily be transferred to the bed supporting member 12, and from there to a mattress and a resting person.

Starting of the drive means may be manually controlled, but may also be sound-controlled such that oscillations start upon detector. detection of a typical sound of snoring by a suitable sound

Although one or more specific drive means may be arranged for imparting vertical oscillating motion (Arrow E) to the bed supporting member 12, it is preferred to let such motion occur spontaneously due to a combination of influence from one or more of the drive means 14-16 and the springs 13. In this regard, it should also be noted that the effect of each crank arm 14b-16b in its respective point of attachment has a horizontal and a vertical component, and that this relatively small vertical component is sufficient to impart a smooth, random vertical oscillating movement to the bed supporting member 12.

1. An oscillating bed supporting structure (10) particularly adapted to provide conditions for a snore-free sleep, said structure comprising a base member (11) and a bed supporting member (12) spaced above said base member (11), at least a first oscillation imparting drive means (14) being provided between said base member (11) and said bed supporting member (12) so as to impart to said bed supporting member (12) oscillating movements (A) at least in a substantially horizontal plane.

2. The oscillating bed supporting structure according to claim 1, characterized by a first (14) and a second (15) separate oscillation imparting drive means imparting to said bed supporting member (12) oscillating movements in mutually substantially perpendicular directions (A and B).

3. The oscillating bed supporting structure according to claim 2, characterized by a third separate oscillation imparting drive means (16) imparting to said bed supporting member (12) oscillating movements (C) in the same direction (A) as one of said first and second oscillation imparting drive means.

4. The oscillating bed supporting structure according to claim 3, characterized in that said first and third oscillation imparting means (14, 16) impart oscillating movement to said bed supporting member (12) at spaced locations along a longitudinal direction (B) of said bed supporting member.

5. The bed supporting structure according to claim 1, characterized in that spring means (13) are interposed between said base member (11) and said bed supporting member (12).

6. The bed supporting structure according to claim 1, characterized in that said drive means (14-16) are adapted to impart oscillating movement to said bed supporting member (12) with variable amplitude and/or variable frequency.

7. The bed supporting structure according to claim 2, characterized in that spring means (13) are interposed between said base member (11) and said bed supporting member (12).

8. The bed supporting structure according to claim 3, characterized in that spring means (13) are interposed between said base member (11) and said bed supporting member (12).

9. The bed supporting structure according to claim 4, characterized in that spring means (13) are interposed between said base member (11) and said bed supporting member (12).

10. The bed supporting structure according to claim 2, characterized in that said drive means (14-16) are adapted to impart oscillating movement to said bed supporting member (12) with variable amplitude and/or variable frequency.

11. The bed supporting structure according to claim 3, characterized in that said drive means (14-16) are adapted to impart oscillating movement to said bed supporting member (12) with variable amplitude and/or variable frequency.

12. The bed supporting structure according to claim 4, characterized in that said drive means (14-16) are adapted to impart oscillating movement to said bed supporting member (12) with variable amplitude and/or variable frequency.

13. The bed supporting structure according to claim 5, characterized in that said drive means (14-16) are adapted to impart oscillating movement to said bed supporting member (12) with variable amplitude and/or variable frequency.
